(19) United States
(12) Patent Application Publication
(10) Pub. No.: US 2008/0306134 A1
(43) Pub. Date: Dec. 11, 2008

(54) THALIDOMIDE ANALOGS FOR TREATING VASCULAR ABNORMALITIES

(75) Inventor: Danyang Chen, Oklahoma City, OK (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: CHARLESSON, LLC

(21) Appl. No.: 11/760,192

(22) Filed: Jun. 8, 2007

(51) Int. Cl. A61K 31/4035; A61P 9/00; C07D 209/48

(52) U.S. Cl. 514/417; 548/476

(57) ABSTRACT

Thalidomide analog compounds having a general structure

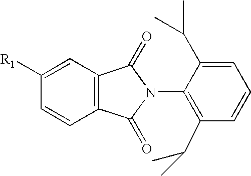

are described. R₁ is selected from a group comprising of hydroxy, hydrogen, and amino. Also described is a method for treating vascular abnormalities, such as neovascularization and vascular leakage. A therapeutically effective amount of a composition containing the thalidomide analog compound is administered to a patient. The composition may further include agents, such as solubilizing agents, inert fillers, diluents, excipients, or flavoring agents.

### BACKGROUND

Since the discovery that thalidomide possessed antiangiogenic activities, thalidomide has been investigated and used experimentally to treat various cancers, dermatological diseases, and inflammatory diseases. It has been found that thalidomide blocked the increase of VEGF in ocular fluid and inhibited the thickening of retinal capillary basement membrane in STZ-diabetic rats, thus representing a potential therapeutic drug for the treatment of diabetic retinopathy. However, thalidomide has also been found to have teratogenic effects, as well as other adverse effects for the treatments of diabetes, for example, producing peripheral neuropathy, hyperglycemia, and imparing insulin action.

Accordingly, there is a need for compounds that have activity as anti-angiogenic agents and can be safely administered to patients to treat angiogenic-associated diseases. The present disclosure is directed to a group of thalidomide analogs and the use of such analogs as inhibitors of angiogenesis.

### SUMMARY

The present disclosure is directed to a compound having a general structure:

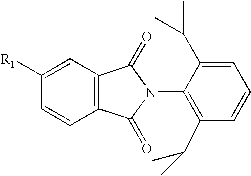

wherein R₁ is selected from a group comprising of hydroxy, hydrogen, and amino.

In accordance with one aspect of the present disclosure, a composition is provided for treating vascular abnormalities in a patient. The composition comprises a compound of the general structure:

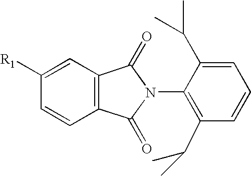

wherein R₁ is selected from a group comprising of hydroxy, hydrogen, and amino. In the preferred embodiment, R₁ is an amino.

In one embodiment, the composition for treating vascular abnormalities further includes at least one agent, wherein the agent is a carrier, solubilizing agent, inert filler, diluent, excipient, or flavoring agent.

In accordance with another aspect of the present disclosure, a method is provided for treating vascular abnormalities in a patient. The method comprises administering to the patient a therapeutically effective amount of a composition. The composition includes a compound of the general structure:

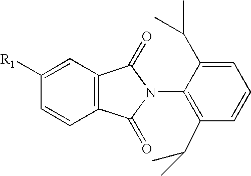

wherein R₁ is selected from a group comprising of hydroxy, hydrogen, and amino. In the preferred embodiment, R₁ is an amino.

In one embodiment, the composition further includes at least one agent, wherein the agent is a carrier, solubilizing agent, inert filler, diluent, excipient, or flavoring agent.

In accordance with another aspect of the present disclosure, a method is provided for synthesizing a compound of a general structure:

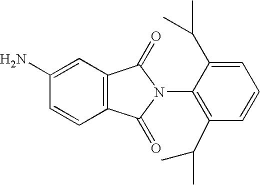

The method comprises providing 5-nitrophthalic anhydrides and 2,4-diisopropylaniline. The 5-nitrophthalic anhydrides and 2,4-diisopropylaniline are refluxed along with acetic acid to form a (2,6-diisopropylphenyl)-5-amino-1H-isoindole-1,3-dione product. The (2,6-diisopropylphenyl)-5-amino-1H-isoindole-1,3-dione product are further refluxed with H₂, Pd/C, and acetone.

### DRAWINGS

The above-mentioned features and objects of the present disclosure will become more apparent with reference to the following description taken in conjunction with the accompanying drawings wherein like reference numerals denote like elements and in which:

FIG. 1 is a table showing the effect of thalidomide and its analogs on cell proliferation.

FIG. 2 is a collection of diagrams showing the inhibition of endothelial cell (HUVEC) migration by Compound 1, Compound 4, and thalidomide.

FIG. 3 is a collection of images showing the effect of Compound 4 on tube formation.

FIG. 4 is a collection of images showing the effect of Compound 4 on blood vessel formation in CAM assay.

FIG. 5 is a collection of images showing the effect of thalidomide analogs on HIF-1α expression.

FIG. 6 is a collection of images showing that Compound 4 down-regulated the expression of VEGF.

FIG. 7 is a collection of graphs showing the effect of thalidomide, Compounds 1, 2, and 4 on retinal vascular leakage in OIR rats.

FIG. 8 is a collection of graphs showing the effect of thalidomide, Compounds 1, 2 and 4 on retinal vascular leakage in STZ-diabetic rats.

FIG. 9 is a collection of images showing retinal angiography of OIR rats with a single intravitreal injection of thalidomide and Compounds 1 and 4.

FIG. 10 is graph showing the rat strain difference in vascular permeability in the OIR model.

FIG. 11 is a collection of graph showing the strain difference in vascular permeability in STZ-diabetic model.

FIG. 12 is a bar graph showing VEGF levels in OIR BN and SD rats.

FIG. 13 is an image showing retinal VEGF levels in BN and SD rats with STZ-diabetes.

FIG. 14 is a collection of graphs showing pharmacokinetic studies of Compound 1.

FIG. 15 is a table showing the effect of Compound 4 on blood vessel formation in CAM assay.

FIG. 16 is a table showing the effect of Compound 4 on the A wave and B wave of eyes in rats.

FIG. 17 is a diagram showing route of synthesis for Compound 4.

FIG. 18 is a collection of diagrams showing the chemical structures of thalidomide and its analogs.

FIG. 19 is a collection of images showing the functional and morphological analysis of the retina treated by Compound 4 in rats.

### DETAILED DESCRIPTION

As used above and elsewhere herein the following terms and abbreviations have the meanings defined below:

AMD Age-related macular degeneration

bFGF Basic fibroblast growth factor

BN Brown-Norway

BSA Bovine serum albumin

CAM Chorioallantoic membrane

CLT003 ([2,6-Diisopropylphenyl])-5-amino-1H-isoindole1,3-dione)/Compound 4

Compound 2 ([2,6-Diisopropylphenyl]-isoindole-1,3-dione

DME Diabetic macular edema

DR Diabetic retinopathy

DMSO Dimethyl sulfoxide

EPO Erythropoietin

ERG Electroretinogram

HIF-1 Hypoxia Induced factor-1

HUVEC Human umbilical vein endothelial cells

IGF-1 Insulin-like growth factor

NV Neovascularization

OIR Oxygen-induced retinopathy

PEG Polyethylene-glycol

PET Polyethylene terephthalate

ROP Retinopathy of prematurity

RPE Retinal pigment epithelial

SD Sprague-Dawley

STZ Streptozotocin

VEGF Vascular endothelial growth factor

VEGFR Vascular endothelial growth factor receptor

The term “angiogenesis” is recognized in the art when used in reference to the generation of new blood vessels into a tissue or organ.

The phrase “therapeutically effective amount” is recognized in the art when used in reference to an amount of the therapeutic agent that produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation.

The term “treatment” is recognized in the art and includes inhibiting or impeding the progress of a disease, disorder or condition and relieving or regressing a disease, disorder, or condition. Treatment of a disease or condition includes ameliorating at least one symptom of the particular disease or condition, even if the underlying pathophysiology is not affected, such as treating the pain of a subject by administration of an analgesic agent even though such agent does not treat the cause of the pain.

The compounds of the present disclosure that have one or more asymmetric carbon atoms may exist as optically pure enantiomers, optically pure diastereomers, mixtures of enantiomers, mixtures of diastereomers, or racemic mixtures of the stereoisomers. The present disclosure includes within its scope all such isomers and mixtures thereof.

The present disclosure relates to novel compounds of thalidomide analogs that have anti-angiogenic activity. More particularly, the disclosure is directed to a series of thalidomide analogs wherein the piperidine-2,6-dione moiety has been replaced with 2, 6-diisopropylaniline as shown below:

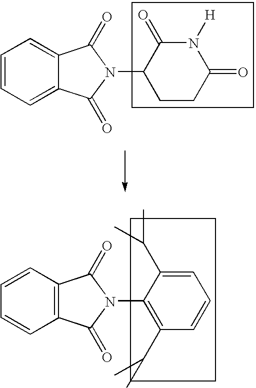

In accordance with one aspect of the present disclosure, a novel compound is provided having a general structure:

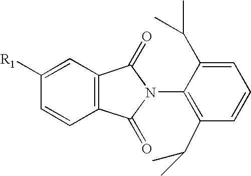

wherein R₁ is selected from a group comprising of hydroxy, hydrogen, and amino. As used above and elsewhere herein, Compound 1 is the embodiment of the compound wherein R₁ is a hydroxy. Compound 2 is the embodiment of the compound wherein R₁ is a hydrogen, and Compound 4 is the embodiment of the compound wherein R₁ is an amino. The various embodiments are shown below:

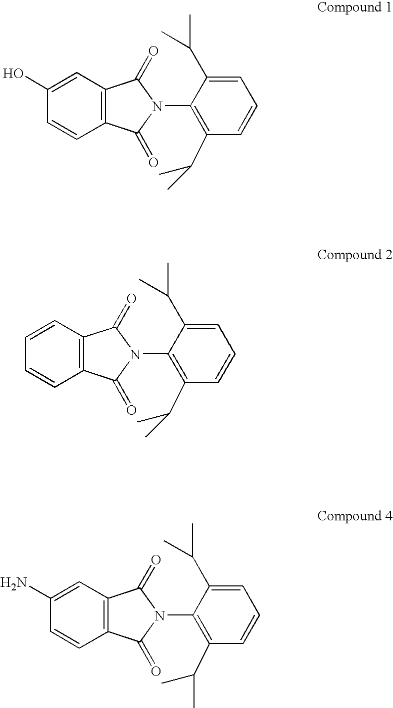

In accordance with a further aspect of the present disclosure, an anti-angiogenic compound is provided having a general structure:

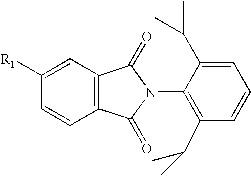

wherein R₁ is selected from a group comprising of hydroxy, hydrogen, and amino.

In a preferred embodiment, the compound has the structure:

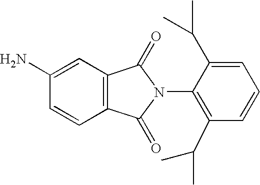

In accordance with another aspect of the present disclosure, a method is provided for treating vascular abnormalities in a patient. More particularly, one embodiment of the disclosure is directed to treating neovascularization and/or vascular leakage. The method comprises administering to the patient a therapeutically effective amount of a composition comprising a compound of the general structure:

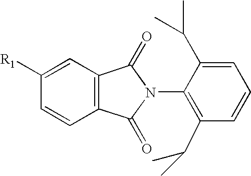

wherein R₁ is selected from a group comprising of hydroxy, hydrogen, and amino.

In one embodiment, the composition is formulated by combining the thalidomide analog compound with one or more agents, which include carriers, solubilizing agents, inert fillers, diluents, excipients, and flavoring agents. As an example, the compound may be incorporated into biodegradable polymers, allowing for sustained release of the compound.

The composition may be administered through various methods to a desired site for treatment, including intravenous, intradermal, subcutaneous, oral (e.g., inhalation), transdermal (i.e., topical), and transmucosal administration. Orally, the composition may be administered as a liquid solution, powder, tablet, capsule, or lozenge. Additives or excipients used in the preparation of tablets, capsules, lozenges and other orally administrate forms may be used in combination with the compound. Parenterally, the composition may be administered, such as through intravenous injection, in combination with saline solutions or conventional IV solutions.

In particular, though not exclusively, the treatment is targeted towards retinal vascular abnormalities, including diabetic retinopathy, diabetic macular edema, age-related macular degeneration, sickle cell retinopathy, retinal vein occlusion, retinopathy of prematurity, and other forms of retinopathy and diseases resulting from retinal neovascularization or retinal vascular leakage. In one embodiment, the treatment includes suppressing VEGF as well as HIF-1α, a major transcription factor up-regulating VEGF in diabetic retina. Additionally, the thalidomide analog compounds may also be used as sodium channel blockers, calcium channel blockers, contraceptives, anti-inflammatory agents and anti-cancer agents.

The thalidomide analog compounds are also anticipated to have use in treating a wide variety of diseases and conditions related to angiogenesis and vascular leakage. The diseases and conditions include, tumors, proteinuria, corneal graft rejection, nonvascular glaucoma and retrolental fibroplasia, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcere, Herpes simplex infections, Herpes zoster infections, protozoan infections, Kaposi sarcoma, Mooren ulcer, Terrien's marginal degeneration, mariginal keratolysis, trauma, rheumatoid arthritis, systemic lupus, polyarteritis, Wegeners sarcoidosis, Scieritis, Steven's Johnson disease, pemphigold radial keratotomy, corneal graph rejection, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma, and post-laser complications.

The dosage of the composition is based on various factors, including the potency of the particular compound, the type of patient (e.g., human or non-human, adult or child), the nature and severity of the disease or condition, the site treated, and the method of administration.

In another aspect of the present disclosure, a thalidomide analog is synthesized by substituting the glutaramide ring with an aromatic group. In one embodiment, Compound 4 is synthesized by using the reactants 5-nitrophthalic anhydrides and 2,6-diisopropylaniline to produce (2,6-diisopropylphenyl)-5-amino-1H-isoindole-1,3-dione, which is further processed to form Compound 4. In a preferred embodiment, 5nitrophthalic anhydrides and 2,6-diisopropylaniline is refluxed with AcOH for 5 hrs to produce (2,6-diisopropylphenyl)-5-amino-1H-isoindole-1,3-dione. (2,6-diisopropylphenyl)-5-amino-1H-isoindole-1,3-dione is further refluxed with H₂, Pd/C, and acetone for 2 hrs to form Compound 4.

### EXAMPLES

A more complete understanding of the present invention can be obtained by reference to the following specific examples and figures. The examples and figures are described solely for purposes of illustration and are not intended to limit the scope of the disclosure. Changes in form and substitution of equivalents are contemplated as circumstances may suggest or render expedient. Although specific terms have been employed herein, such terms are intended in a descriptive sense and not for purposes of limitations. Modifications and variations of the disclosure as hereinbefore set forth can be made without departing from the spirit and scope thereof, and, therefore, only such limitations should be imposed as are indicated by the appended claims.

FIG. 1 is a table comparing the effect of thalidomide and its analogs on cell proliferation.

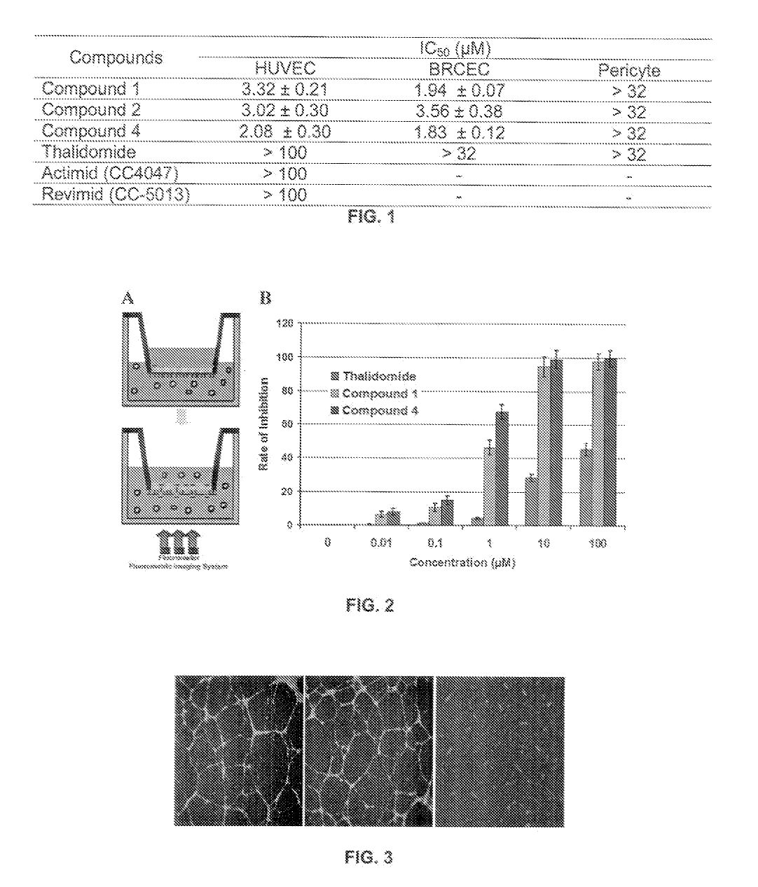

FIG. 2 compares the inhibition of endothelial cell (HUVEC) migration by Compound 1, Compound 4 and thalidomide. FIG. 2A shows a schematic illustration of the Endothelial Cell invasion assay system. In FIG. 2B, cells were seeded at 5×10⁴/insert in EBM-2 containing 0.1% BSA in multi-well inserts. The assembled assays were allowed to proceed for 6 hours. The results are expressed as percent inhibition of migration as compared to control (no inhibitor). Data represents the average of 3 experiments, each run in triplicate. The bars represent mean±SD.

FIG. 3 shows the effect of Compound 4 on tube formation. Representative images were captured after incubation of vehicle, thalidomide and Compound 4 for 16 h. Compound 4 was shown to effectively inhibited tube formation.

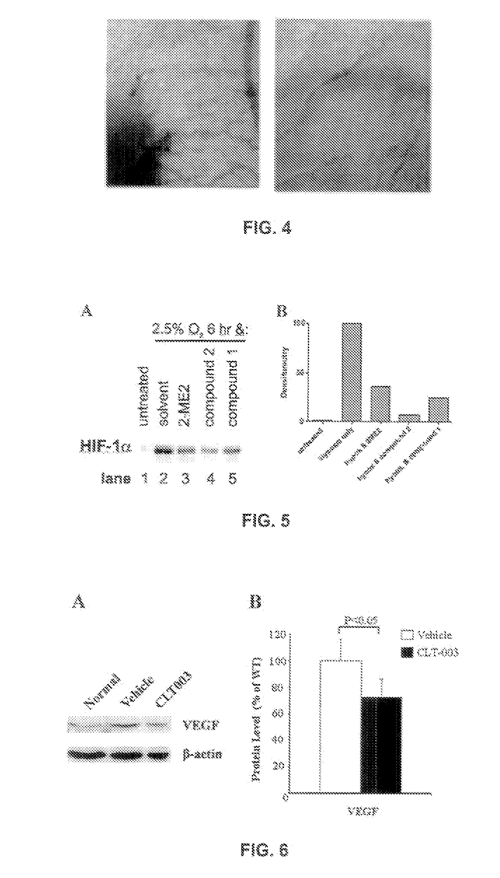

FIG. 4 shows the effect of Compound 4 on blood vessel formation in CAM assay. The left panel in FIG. 4 represents a CAM treated with 200 ng VEGF-165/bFGF for 48 hr. The right panel in FIG. 4 is a representation of a CAM assay treated with 200 ng of VEGF-165/bFGF and 5 μg/embryo of Compound 4.

FIG. 5 shows the effect of thalidomide analogs on HIF-1α expression, HIF-1α in the PC-3 prostate cancer cell treated by hypoxia and compounds was analyzed by western blot (FIG. 5A). Quantitative analysis showed both compound 1 and 2 suppressed hypoxia-induced HIF-1α expression (FIG. 5B).

FIG. 6 shows that Compound 4 (Compound 4) down-regulated the expression of VEGF in the retina of OIR rats. VEGF levels in the retinas from normal rats, vehicle-treated and Compound 4-treated OIR rats was determined by Western blotting (FIG. 6A). FIG. 6B shows the quantitative analysis of VEGF expression. The lane labeled “Normal” represents normal BN rat, “Control” represents intravitreal injection of 5 μl BN rat serum into the left eye, “Compound 4” represents intravitreal injection of 5 μl Compound 4 (0.8 mM in BN rat serum) into the right eye.

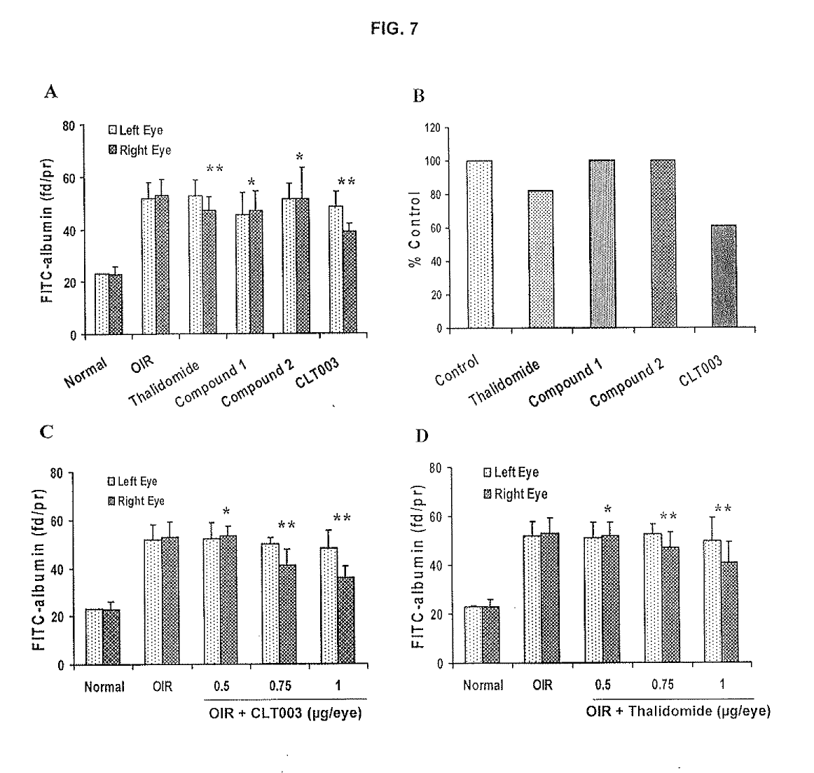

FIG. 7 compares the effect of Compounds 1, 2, 4 (Compound 4) and thalidomide on retinal vascular leakage in OIR rats. In FIG. 7A, OIR rats received an intravitreal injection of 5 μl (0.8 mM in BN rat serum)/eye of thalidomide. Compounds 1, 2, or 4 in the right eye and the same volume of the vehicle in the left eye at P14. Vascular leakage was measured using the FITC-labeled albumin leakage method at P16 and expressed as fd/pr of protein in the retina (mean±SD, n=6). Each of the experimental group was compared with contralateral eye by Student's t test. Retinal vascular leakage in normal non-OIR rats at age of P16 were used as baseline at P16. In FIG. 7B, vascular leakage in the compound-injected eyes was expressed as a percentage of average vascular leakage in the vehicle-injected contralateral eyes. For the control, the average vascular leakage in vehicle-treated retinas was used as 100%. The Thalidomide and Compound 4 reduced retinal vascular leakage by 18% and 40%, respectively (n=6). In FIGS. 7C and 7D, OIR rats received an intravitreal injection of Compound 4 or thalidomide with doses as indicated at P14. Permeability was measured at P16 and expressed as fd/pr of protein in the retina (mean±SD, n=6). Each of the experimental group was compared with the vehicle control by the paired Student's t test, “Normal” is represented as the permeability in normal rats at P16.

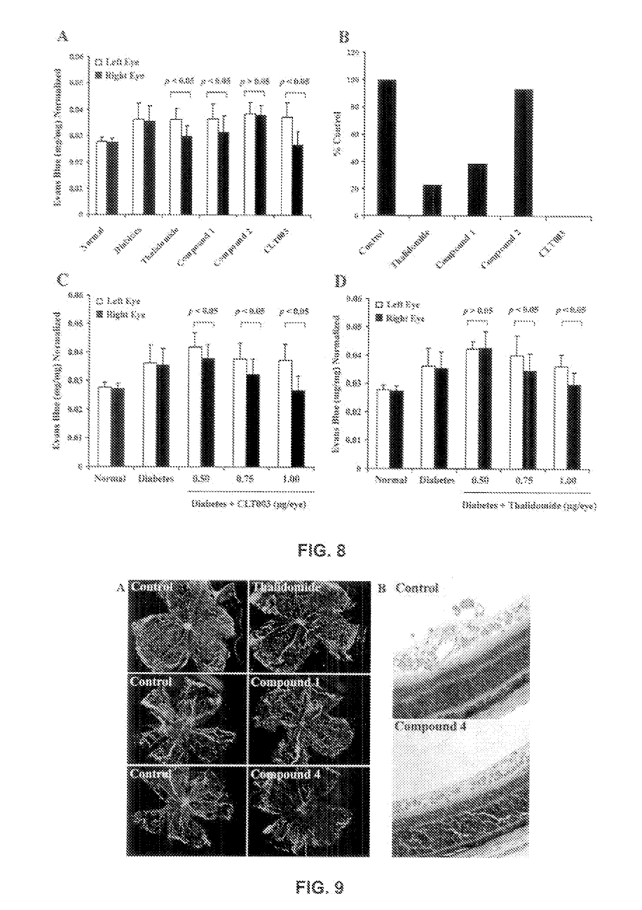

FIG. 8 compares the effect of thalidomide, Compounds 1, 2 and 4 (Compound 4) on retinal vascular leakage in STZ-diabetic rats. In FIG. 8A, two weeks after the induction of diabetes by STZ, diabetic rats received an intravitreal injection of 5 μl (0.8 mM in BN rat serum) per eye of thalidomide, Compounds 1, 2 or Compound 4 into the right eye and the same volume of the vehicle into the left eye. Retinal vascular permeability in the retina was measured by Evans blue-albumin leakage method, 2 days after the injection and normalized by the total protein concentration in the retina and the Evans blue concentration in the blood (mean±SD, n=6). Each of the experimental group was compared with contralateral eyes by Student's t test. Vascular permeability in non-diabetic rats was used as baseline of permeability. “Normal” is represented as the permeability in normal rats at P16. In FIG. 8B, vascular leakage in the compound-injected eyes was expressed as a percentage of that in the vehicle-injected eyes. As the control, STZ-diabetic rats were injected with the vehicle. Thalidomide, Compounds 1, 2 and Compound 4 reduced retinal vascular leakage by 77%, 61% and 100%, respectively (n=6). In FIGS. 8C and 8D, STZ-diabetic rats received an intravitreal injection of Compound 4 or thalidomide with doses as indicated 2 weeks after the induction of diabetes. Permeability was measured 48 h after injection and expressed as mg of Evans blue per mg of protein in the retina (mean±SD, n=6). Each of the experimental group was compared with the vehicle control by the paired Student's t test. “Normal” is represented as the permeability in normal rats.

FIG. 9 shows retinal angiographs of OIR rats with a single intravitreal injection of thalidomide and Compounds. In FIG. 9A, OIR rats received an intravitreal injection of 5 μl of of each compound (0.8 mM in BN rat serum) per eye into the right eye and the same volume of the vehicle into the left eye. Fluorescein retinal angiography was performed at P16. Angiographs are representatives of 3 rats per group. It is to be noted that Compound 4-injected rats have reduced NV, compared to the control. Thalidomide, Compound 1 and 2 did not reduce the NV at the dose used. In FIG. 9B, compared with vehicle-treated rat, the examination of the section showed that pre-retinal NV was decreased in eye treated with Compound 4.

FIG. 10 shows the rat strain difference in vascular permeability in the OIR model. BN and SD rats were treated with hyperoxia and vascular permeability in the retina was measured, normalized by total protein concentration and expressed as percentages of that of respective age-matched normal control (mean±SD, n=4). Values significantly higher than the control are indicated by *.

FIG. 11 shows the strain difference in vascular permeability in STZ-diabetic model. Diabetes was induced in BN and SD rats and permeability in the retina was measured at different time points as indicated. Permeability was normalized by total protein concentrations and expressed as μg of Evans blue per mg of proteins (mean±SD, n=4). Values significantly higher than the age-matched normal control are indicated by *.

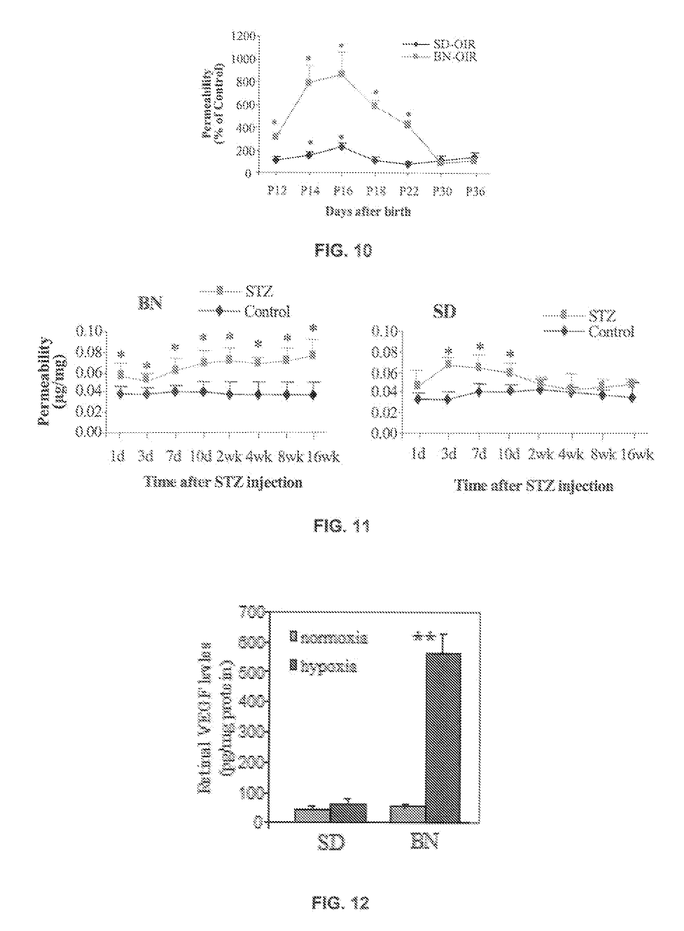

FIG. 12 shows VEGF levels in OIR BN and SD rats. The retinal VEGF levels were measured by ELISA, normalized by retinal protein and expressed as pg/mg protein (mean±SD, n=4). Value significantly higher than the age-matched normal control are indicated by * (P<0.001).

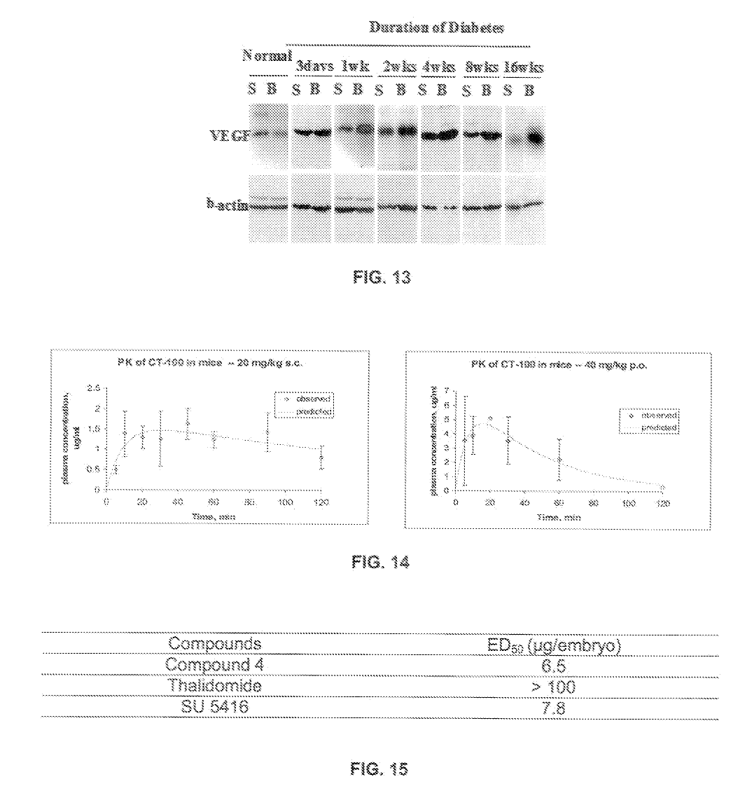

FIG. 13 shows retinal VEGF levels in BN and SD rats with STZ-diabetes. The retinas were dissected from diabetic BN and SD rats at 3 days, and 1, 2, 4, 8 and 16 weeks following the STZ injection. The same amounts of soluble proteins were blotted with an antibody specific to VEGF. The same filter was stripped and re-blotted with anti-β-actin antibody to normalize VEGF levels. The results are from pooled retinas of animals at each point.

FIG. 14 shows the results of pharmacokinetic studies of Compound 1. Mean plasma concentration-time profile of compound 1 in ICR mice after a single dose of subcutaneous dosing (A, 20 mg/Kg) and oral dosing (B, 40 mg/Kg). Each data point represents the mean±standard deviation of 10 mice.

FIG. 15 is a table comparing the effect of Compound 4 on blood vessel formation. Thalidomide and SU5416 on dose in μg/embryo of compound necessary to reduce the blood vessel number to 50% that of the VEGF/bFGF alone group, a level of blood vessels similar to the untreated “control” group. Thus thalidomide alone has an apparent ED₅₀ of >100 μg/embryo, Compound 4 and SU5416 has an apparent ED50 of 6.5 and 7.8 μg/embryo, respectively. Data represent mean±SD of 8-16 samples from 2-3 separate experiments.

FIG. 16 is a table comparing the effect of Compound 4 on the A wave and b wave of eyes in rats.

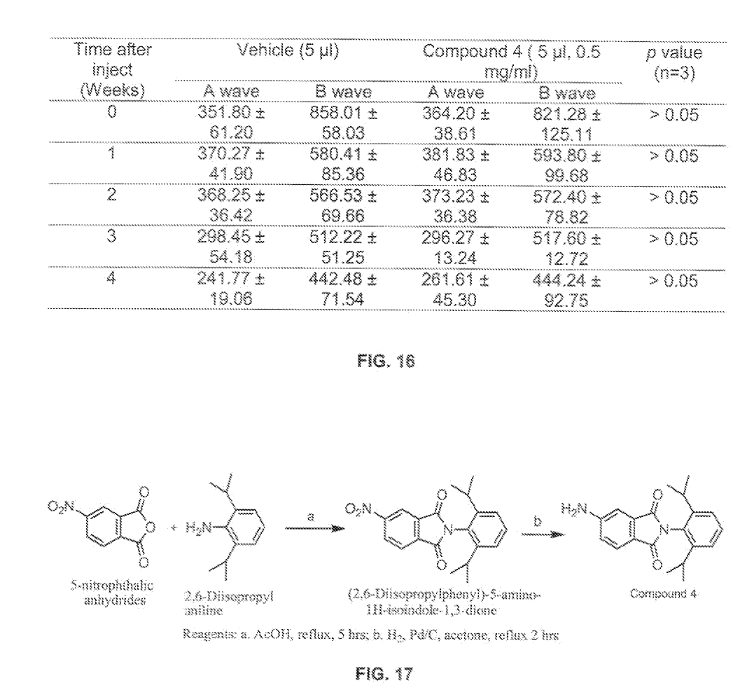

FIG. 17 is a diagram showing the route of synthesis for Compound 4. ¹H-NMR (250 MHz, CDCl₃) δ 1.08 (12H, d, J=6.80 Hz), 2.50 (2H, hept, J=6.80 Hz), 6.75 (1H, dd, J=1.98 Hz, 8.25 Hz), 6.87 (1H, d, J=1.98 Hz), 7.16 (2H, d, J=7.85 Hz), 7.31 (1H, t, J=7.85 Hz), 7.46 (1H, d, J=8.25 Hz), mp 252-253° C. (lit. 253-254° C.).

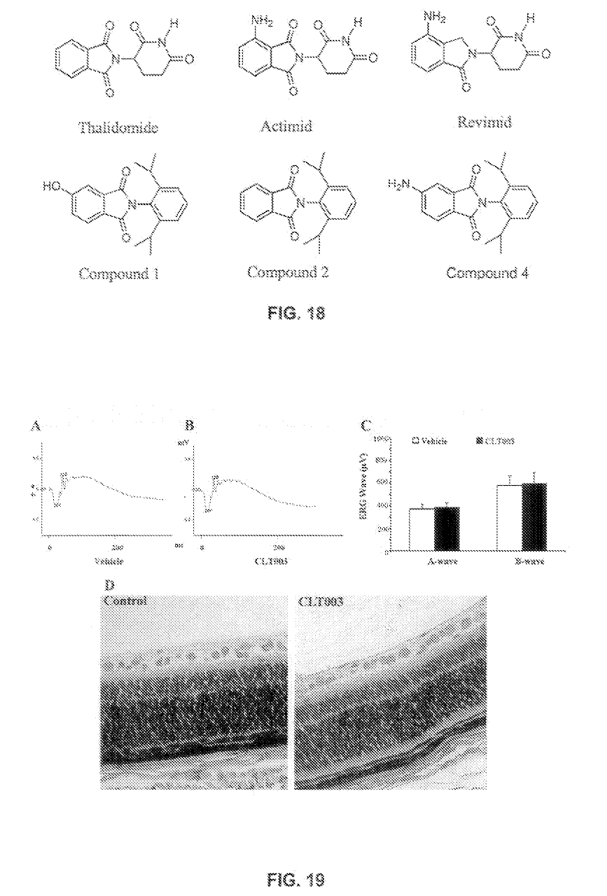

FIG. 18 is a diagram of the various chemical structures of thalidomide, actimid, revimid, Compound 1, 2, and 4.

FIG. 19 shows the functional and morphological analysis of the retina treated by Compound 4 in rats. Eight weeks old BN rats were received an intravitreal injection of Compound 4 (2.0 μg/eye, 5 μl/eye 0.4 mg/ml in BN rat serum) or equal amount of BN rat serum respectively (n=6). ERG was performed prior to study initiation and 1, 2, 3 and 4 weeks after the injection. Data shown no dramatic change in the a-wave and b-wave amplitudes in Compound 4-injected rats compared to vehicle-injected rats (FIG. 19A-19C). The animals were sacrificed 4 weeks after injection. The eye sections were observed under microscope with HE staining. Pathological observation showed that no detectable morphological change was found in the retinas of rats treated by Compound 4 and control (FIG. 19D).

Materials and Methods

Cell culture: All cell culture media and supplements were purchased from Cellgro unless otherwise indicated. Human Umbilical Vein Endothelial Cells (HUVEC) were obtained from American Type Culture Collection and grown in the EBM-MV2 medium (Clonetics). Bovine Retinal Endothelial Cells (BREC) and pericytes were isolated according to a modified method as described previously (Wong, et al. Investig. Opthalmol. Vis. Sci. 1987, 28: 1767-1775). Twelve bovine eyes were obtained from a local slaughterhouse (Country Home Meats). The retinas were removed and washed four times in DMEM. Subsequently retinas were homogenized and centrifuged at 400×g for 10 min. The resultant pellet was resuspended in an isolation medium (DMEM with 100 IU/ml penicillin, 100 μg/ml streptomycin and 250 ng/ml amphotericin). Microvessels were trapped on an 85 μm nylon mesh (Locker Wire Weavers LTD) and transferred to a petri dish (Falcon) containing 10 ml of an enzyme cocktail which consisted of 600 μg/ml DNase I (Sigma), 165 μg/ml collagenase (Sigma) and 700 μg/ml Pronase E (EMD) and were incubated at 37° C. for 20 min. The resultant vessel fragments were trapped on a 53 μm nylon mesh (Locker Wire Weavers LTD), washed with the isolation medium and centrifuged at 400×g for 5 min. For selective culture of pericytes, the resultant pellet was resuspended in 10 ml of the pericyte growth medium and transferred into 75-cm² plastic tissue culture flasks (BD Biosciences). For selective culture of BRCECs, the resultant pellet was resuspended in 10 ml of the BRCEC growth medium and transferred into 75-cm² collagen-coated plastic tissue culture flasks (BD Biosciences). The BRCEC growth medium consisted of DMEM supplemented with 10% human serum, 1% glutamine, 1 mg/ml insulin, 550 μg/ml transferring, 670 ng/ml selenium, 100 IU/ml, penicillin, 100 μg/ml streptomycin, 250 ng/ml amphotericin, 90 μg/ml heparin (Sigma) and 15 μg/ml endothelial cell growth supplement (Upstate). Cells were cultured at 37° C. and 5% CO₂ with regular medium change every 3 days. Confluence cultures were passaged by detaching the cells with 0.25% trypsin and plated at a split 1:3. Purity of BRCECs and pericytes were confirmed by binding of Dil-Ac-LDL (Biomedical Technologies Inc) to LDL receptor on the surface of BRCECs and immunolabeling with anti-smooth muscle antibody (Sigma), respectively. At passage 2, BRCECs and pericytes were stored in a liquid nitrogen tank for future use.

MTT assay: Cells were seeded at a density of 5×10⁴ cells per well in 400 μl of growth medium in triplicate in 24-well plates (Nalge Nunc) or gelatin-coated 24-well plates. Twenty-four hours after seeding, the growth medium was replaced by a medium containing 1% FBS, with or without different concentrations of thalidomide or thalidomide analogs. After the cells were treated for 48-72 h, MTT was added to a final concentration of 0.5 mg of medium per ml and incubated for 4 h at 37° C. in 5% CO₂. An equal volume of solubilizer buffer is then added, following the protocol recommended by the manufacturer (Roche Molecular Biochemicals), the cells will be incubated overnight at 37° C. in 5% CO₂. The absorbance of the formazen product was measured at a wavelength of 570 nm, with 750 nm as the (subtracted) reference wavelength.

Endothelial cell migration assay: The fluorescence-based endothelial cell invasion assay used a BD Matrigel™ and BD Falcon™ HTS FluoroBlok™ (BD Biosciences) 24-Multiwell Insert System (FIG. 2A). The insert system consisted of fluorescence-blocking 3 μm PET membrane, which blocks light transmission at wavelengths 490-700 nm, sealed to multiwell inserts. This made it possible to directly measure fluorescent signal from cells that had undergone invasion through Matrigel to the bottom side of inserts by using signal from cells that had undergone invasion through Matrigel to the bottom side of inserts by using bottom reading mode of a fluorometer. In this assay system, HUVECs were allowed to invade in the absence (control) or presence of VEGF (4 ng/ml) with varying concentrations (0.01-100 μM) of Compounds 1, Compound 4 and thalidomide in the bottom. Cells were allowed to invade for 22±1 hours. Cells were labeled post invasion with Calcein AM (4 μg/ml) and measured by detecting the fluorescence of cells that invaded through the BD Matrigel™ Matrix with an Applied Biosystems CytoFluor® 4000 plate reader at 485 nm excitation and 530 nm emission.

Chicken chorioallantoic membrane (CAM) assay: The fertile leghorn chicken eggs were incubated in a humidified environment at 37.5° C. for 10 days. The human VEGF-165 and basic fibroblast growth factor (bFGF) (200 ng each) were then added to saturation to a microbial testing disk and placed onto the CAM by breaking a small hole in the superior surface of the egg. Anti-angiogenic compounds were then added 8 hr after the VEGF/bFGF at saturation to the same microbial testing disk, and the embryos were incubated for an additional 40 h. CAMs were then removed, quickly fixed with 4% paraformaldehyde in PBS, placed onto Petri dishes, and digitized images taken at 7.5× using a Nikon dissecting microscope and Scion Imaging system. A 1×1-cm grid was then added to the digital CAM images and the average number of vessels within 5-7 grids counted as a measure of vascularity.

Induction of oxygen-induced retinopathy (OIR): Induction of OIR followed the procedure as described by Smith et al (Smith, et al. Invest Ophthalmol. Vis. Sci. 1994, 35: 101-111) with some modifications. Briefly, Newborn Brown Norway (BN) rats (Charles River Laboratories) at postnatal day 7 (P7) were exposed to hyperoxia (75% O₂) for 5 days (P7-12) and then returned to normoxia (room air) to induce retinopathy.

Induction of diabetes by streptozotocin (STZ); BN rats (8 weeks of age) were given a single intraperitoneal injection of fresh made streptozotocin (STZ) (Sigma, 50 mg/kg in 10 mM of citrate buffer, pH 4.5) following an overnight fasting. Control rats received an injection of citrate buffer alone. Blood glucose levels were checked at 24 hours following the last STZ injection and once a week thereafter, and only the animals with glucose levels higher than 350 mg/dl were considered diabetic. Rats with hyperglycemia for 2 weeks were used for these experiments.

Intravitreal injection of compounds: Thalidomide and its analogs Compounds 1, 2 and Compound 4 were dissolved in vehicle (BN rat serum) and sterilized by filtration. OIR and STZ-diabetic BN rats received an intravitreal injection of 0.5-2.0 μg/eye of (5 μl/eye, 0.1-0.4 mg/ml in BN rat serum) of thalidomide, Compounds 1, 2 or Compound 4 into the right eye and the equal volume of the BN rat serum into the left eye.

Retinal angiography with high-molecular-weight fluorescein: High molecular weight fluorescein-dextran was used in retinal angiography as described by Smith et al (Smith, et al. Invest. Ophthalmol. Vis. Sci. 1994, 35:101-111). Briefly, animals were anesthetized with ketamine (100 mg/kg of body weight) plus acepromazine (5 mg/kg of body weight) and then perfused through the left ventricle with 50 mg/ml of high molecular weight fluorescein-dextran in PBS. The eyes were marked for orientation, enucleated, and fixed in 4% paraformaldeyde for 3-24 h. Several incisions were made and the retinas were flat-mounted on a gelatin-coated slide. The vasculature was then examined under a fluorescent microscope. Both the total retinal area and the area of the avascular regions were measured using a computerized image-analysis system and averaged within each group.

Measurement of vascular permeability: Vascular permeability was quantified by measuring leakage of FITC-albumin or Evans blue dye-albumin complex from the blood vessels into the retina as described (Xu, et al. Invest. Ophthalmol. Vis. Sci. 2001, 42:789-794), with some modifications. Briefly, FITC-albumin was injected through the femoral vein and circulated for 2 h. The rats were then perfused via the left ventricle. The retinas were carefully dissected and homogenized. The concentrations of FITC-albumin were measured in a fluorometer and normalized by the total protein concentration in each retina and by plasma concentration of FITC-albumin.

Evans blue dye (Sigma) was dissolved in 0.9% saline (30 mg/ml), sonicated for 5 min and filtered through a 0.45-μm filter (Millipore). The rats were then anesthetized, and Evans blue (30 mg/kg) was injected over 10 s through the femoral vein using a glass capillary under microscopic inspection. Evans blue non-covalently binds to plasma albumin in the blood stream. Immediately after Evans blue infusion, the rats turned visibly blue, confirming their uptake and distribution of the dye. The rats were kept on a warm pad for 2 h to ensure the complete circulation of the dye. Then the chest cavity was opened, and the rats were perfused via the left ventricle with 1% paraformaldehyde in citrate buffer (pH 4.2) which was pre-warmed to 37° C. to prevent vasoconstriction. The perfusion lasted 10 min under the physiological pressure of 120 mmHg, in order to clear the dye from the vessel. Immediately after perfusion, the eyes were enucleated and the retinas were carefully dissected under an operating microscope. Evans blue dye was extracted by incubating each sample in 150 μl of formamide for 18 h at 70° C. The extract was centrifuged (Beckman) at 70,000 rpm (Rotor type: TLA 100.3) for 20 min at 4° C. Absorbance was measured using 100 μl of the supernatant at 620 nm by using Spectrophotometer DU800 (Beckman). The concentration of Evans blue in the extract was calculated from a standard curve of Evans blue in formamide and normalized by the total protein concentration in each sample. Results were expressed in mg of Evans blue per mg of total protein content.

Immunolabeling: Cultured cells were immediately fixed in 4% paraformaldehyde in 1× PBS for 10 min, washed in PBS three times for 5 min, and blocked in 0.5% BSA for 20 min. Washing cells in PBS three times before and after a 1 hr primary antibody incubation was followed by staining for 1 hr with secondary antibodies. The immunolabeling signals were subsequently detected by incubating cells with FITC or Texas red-conjugated secondary antibodies (Jackson Immunoresearch). Coverslips were washed in PBS and stained with 0.2 μg/ml DAPI prior to mounting. Fluorescent images were collected on a Zeiss fluorescent microscope or a Zeiss 510 confocal laser scanning microscope equipped with an argon-krypton laser.

Western blotting: Proteins were extracted by incubating in a lysis buffer. Equal amounts of proteins from different samples were separated by SDS-PAGE for Western blot analyses using an antibody directed against VEGF. Immunobloting signals were visualized by conversion of SuperSignal West Pico Chemiluminescent Substrate (Pierce).

Electroretinogram (ERG) recording: Full-field ERGs were recorded by Espion E² ERG system (Diagnosys LLC) as described previously (Rohrer, Journal of Neuroscience, 1999, 19: 8919-8913) by two protocols; (A) 10 ms flashes of increasing light intensities under scotopic and photopic conditions, and (B) 2 Hz flicker ERG under photopic conditions. BN rats received an intravitreal injection of Compound 4 (2.0 μg/eye, 5 μl/eye of 0.4 mg/ml in BN rat serum) or equal amount of BN rat serum, respectively. At various intervals after injection, the peak a-wave amplitude was measured from baseline to the initial negative-going voltage, whereas peak b-wave amplitude was measured from the trough of the a-wave to the peak of the positive b-wave. Flicker amplitudes were measured from the preceding trough to the peak of the flicker response. Data was expresses as mean±SD and compared between the compound-injected eyes and control eyes by the paired Student's t test.

Histological analysis of the retina: To test the potential ocular toxicity of Compound 4, 8 week old normal BN rats received an intravitreal injection of Compound 4 (2.0 μg/eye, 5 μl/eye of 0.4 mg/ml in BN rat serum) or equal amount of BN rat serum, respectively. At various intervals after injection, the animals were sacrificed. The eyes then were removed, fixed in 4% formaldehyde, embedded in paraffin, and cut into 6-μm sections containing the whole retina. Paraffin-embedded sections were stained with hematoxylin-eosin (HE) and were examined.

Experimental Results

A series of novel thalidomide analogs have been designed, synthesized and experimentally tested. The discoveries and results of the experiments are included below:

Experiment 1: Compound 4 was Found to be Substantially More Potent Than Thalidomide and the Other Two Analogs in Inhibition of Proliferation of Endothelial Cells.

Primary endothelial cells (HUVEC and BRCEC) and pericytes were treated with various concentrations of the compounds for 3 days. Viable cells were quantified using MTT assay and IC₅₀ of each compound was calculated (mean±SD, n=3, FIG. 1). The IC₅₀ values represent the means and SE of 3 independent experiments. Compounds 1, 2 and Compound 4 inhibited the proliferation of endothelial cells in a dose-dependent manner with an IC₅₀ of 3.3, 3.0 and 2.0 μM, respectively, for HUVECs and 1.94, 3.56 and 1.83 μM, respectively, for BRCECs. Thalidomide had weaker effects with IC₅₀>100 μM in HUVECs and with IC₅₀>32 μM in BRCECs (FIG. 1). Exiting thalidomide analogs, Actimid (CC4047) and Revimid (CC-5013), had weaker effects with IC₅₀>100 μM in HUVECs. Under the same conditions, these compounds did not significantly inhibit pericyte growth, suggesting specific inhibition to endothelial cells. These results indicated that Compound 4 had more potent anti-angiogenic effects than the other 2 compounds and thalidomide.

Experiment 2: Compound 4 was Found to Have a More Potent Inhibitory Effect Than Thalidomide on Migration of HUVEC.

The effect of Compound 1, Compound 4, and thalidomide on endothelial migration was evaluated using in vitro migration (invasion) assay. The advantage of this assay is that it can be amended to high-throughput screening. It is a fluorescence-based endothelial cell invasion assay system. This assay system is based on BD Matrigel™ and BD Falcon™ HTS FluoroBlok™ (BD Biosciences, Bedford, Mass.) 24-Multiwell Insert System (FIG. 2A). The insert system consists of fluorescence-blocking 3 μm PET membrane, which blocks light transmission at wavelengths 490-700 nm, sealed to multiwell inserts (FIG. 2A). This makes it possible to directly measure fluorescent signal from cells that have undergone invasion through Matrigel to the bottom side of inserts by using signal from cells that have undergone invasion through Matrigel to the bottom side of inserts by using bottom reading mode of a fluorometer. In this assay system, human endothelial cells are allowed to invade and are then labeled with fluorescent dye Calcein AM before quantification on a fluorometer. Compound 1 and Compound 4 inhibited the endothelial cell migration and showed the dose response curves with an IC₅₀ of 1 μM and <1 μM. Thalidomide, on the other hand, exhibited an IC₅₀ of >100 μM, as shown in FIG. 2B.

Experiment 3: Compound 4 was Found to Inhibit Tube Formation From Endothelial Cells.

Eight-well slide chambers were coated with matrigel and at 37° C. and 5% CO₂ for 30 min. HUVECs were then seeded at 30,000 cells/well in EGM-II containing either vehicle (0.5% DMSO), 5 μM of Compound 4 or thalidomide and incubated at 37° C. and 5% CO₂ for 16 h. After incubation, slides were washed in PBS, fixed in 100% methanol for 10 s, and stained with DiffQuick solution II for 2 min. To analyze tube formation, each well was digitally photographed using a ×2.5 objective. The tube formation assay showed the qualitative representative images of the potency of Compound 4 on inhibition of tube formation. On the contrary, thalidomide did not show any inhibitory activity as shown in FIG. 3.

Experiment 4: Compound 4 was Found to Fee More Potent Than Thalidomide and Compounds 1 and 2 in Inhibiting Vascular Formation in the CAM Assay.

CAM assay was used for in vivo anti-angiogenic studies. The fertile leghorn chicken eggs were allowed to incubate in a humidified environment at 37.5° C. for 10 days. The human VEGF-165 and bFGF (200 ng each) were then added to saturation to a microbial testing disk and placed onto the CAM by breaking a small hole in the superior surface of the egg. Anti-angiogenic compounds were then added 8 hours after the VEGF/bFGF at saturation to the same microbial testing disk and embryos allowed to incubate for an additional 40 hours. After 48 hr, CAMs wore removed, quickly fixed with 4% paraformaldehyde in PBS, placed onto Petri dishes, and digitized images taken at 7.5× using a Nikon dissecting microscope and Scion Imaging system. A 1×1-cm grid was then added to the digital CAM images and the average number of vessels within 5-7 grids counted as a measure of vascularity. FIG. 4 shows a representative CAM treated with VEGF-165/bFGF for 48 hr and a CAM treated with VEGF/bFGF and 5 μg of Compound 4 for 48 hr. VEGF/bFGF induced CAM blob vessel formation. At 5 μg/embryo, Compound 4 was able to inhibit CAM blood vessel formation induced by VEGF/bFGF. Compound 4 has an ED₅₀ of 6.5 μg/embryo, while thalidomide has an apparent ED₅₀ of >100 μg/embryo, suggesting Compound 4 inhibited blood vessel formation in the CAM assay (FIG. 15).

Experiment 5: Thalidomide Analogs were Found to Suppress Hypoxia-Induced HIF-1α Production in PC-3 Prostate Cancer Cells.

Suppression of hypoxia-induced HIF-1α expression by Compound 1, Compound 2 and 2ME2 (positive control) was tested in PC-3 prostate cancer cells. Cells were exposed to 10 μM (containing 0.1% DMSO) of inhibitors or DMSO alone as control overnight, HIF-1α expression in the PC-3 prostate cancer cell treated by hypoxia and compounds was analyzed by western blotting (FIG. 5A). Both compound 1 and 2 significantly suppress hypoxia-induced HIF-1α expression by 79-90% (FIG. 5B).

Experiment 6: Compound 4 was Found to Down-Regulate the Expression of VEGF in the Retina of OIR Rats.

VEGF is believed to play a critical role in DME. HIF-1α regulates transcriptional activation of VEGF in response to hypoxia. The tested thalidomide analogs significantly suppressed hypoxia-induced HIF-1α expression in vitro studies, suggesting that these compounds may reduce retinal vascular leakage through VEGF signaling. To address the hypothesis, the expression of VEGF in the Compound 4-injected OIR rats was determined. Proteins of retinas from normal rats, vehicle-treated and Compound 4-treated OIR rats were extracted by incubating and sonicating in lysis buffer. Equal amounts of proteins from each samples were separated by SDS-PAGE for Western blot analyses using antibody directed against VEGF. Immunoblotting signals were visualized by conversion of SuperSignal West Pico Chemiluminescent Substrate (Pierce). The result has shown that the expression of VEGF decreased in retina of Compound 4-treated OIR rat (FIG. 5).

Experiment 7: Compound 4 was Found to Have a More Potent Effect on Retinal Vascular Leakage in OIR Rats After an Intravitreal Injection.

To induce OIR, BN rats at postnatal day 7 (P7) were exposed to hyperoxia (75% O₂) for 5 days (P7-P12) and then returned to normoxia. Normal control rats were kept in room air. At P14, the OIR BN rats received an intravitreal injection of 5 μl (0.8 mM in BN rat serum)/eye of thalidomide, Compound 1, 2 or Compound 4 into the right eye and same volume of the BN rat serum into the left eye. Retinal vascular leakage was measured using FITC-labeled albumin as tracer. Normal non-OIR BN rats (n=6) served as baseline at P16. At P16, retinal vascular leakage decreased in the thalidomide-treated eyes to 82% of the contralateral eyes injected with vehicle (paired t test, P<0.05, n=6). Compound 4 decreased the retinal vascular leakage to 61% of the contralateral control (paired t test, P<0.05, n=6). At the same concentration, Compounds 1 and 2 did not significantly reduce the retinal vascular leakage (FIGS. 7A and 7B). Fluorescein angiography showed that Compound 4 had weak effect on retinal NV at the dose used (FIG. 9), suggesting that Compound 4 induced reduction of retinal vascular leakage is more potent than its effect on retinal NV.

To determine if the effect of Compound 4 on retinal vascular leakage was dose-dependent, the OIR rats at P14 received a single injection of Compound 4 with doses of 0.5, 0.75 and 1.0 μg/eye (5 μl of 0.10, 0.15 and 0.20 mg/ml). Compound 4 and thalidomide significantly reduced vascular leakage at doses of 0.75 and 1.0 μg/eye (p<0.05, n=6) but not at the dose of 0.5 μg/eye (FIGS. 7C and 7D), indicating a dose-dependent effect on vascular leakage in OIR rats.

Experiment 8: Compound 4 was Found to Have a More Potent Effect on Retinal Vascular Leakage in STZ-Diabetic Rats.

Diabetes was induced by injection of STZ (50 mg/kg, i.v.) into adult BN rats after overnight fasting. Blood glucose levels were monitored at the second day after the injection and once a week thereafter. Rats with glucose levels above 350 mg/dl were considered as diabetic and used for the study. Thalidomide, Compounds 1, 2 and 4 were separately injected into the vitreous space (5 μl, 0.8 mM in BN rat serum) of the right eye of STZ-diabetic rats 2 wks after the induction of diabetes. At 48 h after the injection, retinal vascular leakage was measured using the Evans blue-albumin leakage method. The result showed that the eyes injected with thalidomide, Compound 1 and Compound 4 had a significant reduction in vascular leakage in the retinas, compared to the contralateral eyes injected with the vehicle (P<0.01, n=6) (FIG. 8A). Thalidomide reduced vascular leakage by 77%, Compound 1 reduced vascular leakage by 61%, and Compound 4 reduced vascular leakage by almost 100% (FIG. 8B), to normal level (baseline), suggesting that Compound 4 completely blocks the retinal vascular leakage. To determine the time course of the effect of Compound 4 after intravitreal injection, OIR rats received 5 μl (0.8 mM in BN ml serum)/eye of Compound 4 into the right eye at P14. 24 h and 48 h after administration, retinal vascular permeability measurements showed that the Compound-injected eye had completely been blocked in comparison with the control of the contralateral eye.

To determine the dose-response relationship of the effect of Compound 4 and thalidomide, the STZ-diabetic rats received an intravitreal injection of Compound 4 and thalidomide with doses of 0.5, 0.75 and 1.0 μg/eye (5 μl/eye of 0.10, 0.15 and 0.20 mg/ml), respectively. Two days after the injection, Compound 4, at all of these doses significantly reduced vascular permeability in the retina, when compared to the vehicle control (P<0.05, n=6) (FIG. 8C). However, thalidomide showed an inhibitory effect only at the doses of 0, 75 and 1.0 μg/eye (P<0.05, n=6), but not at 0.5 μg/eye (p>0.05, n=6) (FIG. 8D). This observation indicates that Compound 4 has more potent effect on reducing retinal vascular leakage not only in the OIR model but also in the experimental diabetes model, compared to thalidomide and the other compounds.

Experiment 9: Compound 4 was Found to Have an Inhibitory Effect on Retinal NV in the OIR Model.

Newborn BN rats were exposed to 75% oxygen from age P7 to P12. The rats were then kept in room air for 4 days to allow partial formation of retinal NV. At age P16 when retinal NV has formed partially, OIR rats received a single intravitreal injection of thalidomide and Compounds 1, 2, and Compound 4 of 1.0 μg/eye (5 μl/eye of 0.2 mg/ml in BN rat serum) into the vitreous of the right eye and the vehicle (5 μl BN rat serum) into the left eye for control. Retinal NV was evaluated at age P20 by fluorescein angiography in flat-mounted retinas. The retinal vasculature was visualized under a fluorescent microscope and compared with that in the contralateral control eye (FIG. 9A). The neovascular events were observed on eye sections (FIG. 9B). Results displayed that Compound 4 partly inhibited the retinal NV in OIR rats, while Compound 1, 2, and thalidomide lacked significant inhibition of retinal NV in OIR rats.

Experiment 10: Rat Strain Difference in Vascular Leakage in the Retinas of OIR and STZ-Induced Diabetic Rats.

A model was established for sustained retinal vascular leakage for testing the long-term effect of new drugs. The time courses of retinal vascular permeability were defined in both the OIR and STZ-diabetic models in Sprague Dawley and BN rats. OIR was induced by exposing neonatal rats to hyperoxia (75% O₂) from P7 to P12. Diabetes was induced in adult BN rats by STZ injection. Retinal vascular permeability was measured using the Evans Blue-albumin method. In OIR-BN rats, the permeability started to increase at P12, reaching its peak at P16 with an 8.7-fold increase over the level in age-matched normal rats (P=7.5E−06). Between P18 and P22, the permeability slowly declined, reaching normal levels after P30 (FIG. 10). In OIR-SD rats, the permeability started to increase later (P14). The peak value was lower than that in BN rats (2.2-fold) and permeability declined to the normal level by P18 (FIG. 10). These observations correlated with different retina VEGF levels in the two strains. In STZ-BN rats, hyper-permeability occurred 24 h after the STZ injection (1.4-fold; P=0.0292) and reached a plateau at 2 wks (1.8-fold, P=0.0074). The hyper-permeability lasted at least 16 wks after the induction of diabetes. In STZ-SD rats, the permeability started to increase 3 days after the STZ-injection (1.3-fold; P=0.0271), reached its peak at 1 wk (1.5-fold: P=0.004) and declined to the control level by 2 wks (FIG. 11). These results suggest that in both OIR and STZ-diabetes, vascular leakage is significantly higher and lasts longer in BN than in SD rats. Therefore, all of the studies in this project involving rat models used BN rats. These results also suggest that the OIR model is good for short term effect while the STZ-diabetes model is suitable for evaluating long-term effect of Compound 4 on retinal vascular leakage as proposed in this Phase II project.

Experiment 11: BN Rats were Found to Have Higher VEGF Levels in the Retina Than SD Rats in Response to Ischemia.

To determine if the more severe retina NV in BN rats are correlated with their retinal VEGF over-production in the OIR model, VEGF levels were quantified using a rat VEGF ELISA kit (R&D systems, Inc) and normalized by total retinal protein concentrations. The results showed that the basal level of retinal VEGF were similar in normal BN and SD rats. In OIR-SD rats, retinal VEGF levels had no significant difference compared with those in normal control SD rats (FIG. 12). However, retinal VEGF levels in OIR-BN rats were about 10-folds higher than those in normal control BN and OIR SD rats (P<0.001, n=4) (FIG. 12).

Experiment 12: BN Rats were Found to Have Higher VEGF Induction in the Retina Than SD Rats in Response to STZ-Induced Diabetes.

Studies have shown that BN rats with STZ-induced diabetes develop more severe retinal vascular leakage than STZ-diabetic SD rats with similar hyperglycemia and duration. To determine if the retinal VEGF expression is up-regulated more significantly in BN than in SD rats by diabetes, retinal VEGF levels were measured and semi-quantified by Western blot analysis in BN and SD rats with STZ-induced diabetes and compared to respective age-matched non-diabetic controls at different time points after the onset of diabetes. The results showed that the basal level of retinal VEGF expression was similar in normal adult BN and SD rats (FIG. 11). Following the induction of diabetes by STZ, however, the retinal VEGF levels in diabetic BN rats were higher than those in diabetic SD rats during the time period of 3 days to 16 weeks of diabetes (FIG. 13).

These observations suggest that the retinas of BN rats with OIR or STZ-diabetes are suitable in vivo models for investigating the mechanism of Compound 4, i.e., its effect on VEGF over-expression.

Experiment 13: Pharmacokinetic Studies of Compound 1.

Preliminary pharmacokinetic studies of Compound 1 were performed through subcutaneous and oral dosing. Animals used in the study were ICR mice weighing about 30 g. A subcutaneous dose of 20 mg/kg body weight or oral dose of 40 mg/kg body weight were given to the animals. Compound 1 was dissolved in PEG 300 to final concentration of 5 mg/ml (for s.c.) or 10 mg/ml (for p.o.). Blood samples were obtained by retro-orbital sinus puncture under isoflurane anesthesia and were collected at 5, 10, 20, 30, 45, 60, 90, 120 minutes after subcutaneous dose. After oral dose by gavage, blood samples were collected at 5, 10, 20, 30, 45, 60, 90, 120 minutes later. Blood samples were kept on ice until centrifuged at 16,000×g at 4° C. for 10 minutes. Plasma fraction was collected and stored at −20° C. until analysis. Upon analysis 200 μl of plasma was spiked with 20 μl of 100 μg/ml internal standard, and 450 μl of acetonitrile was added to each tube, then centrifuge at 16,000×g at 4° C. for 10 minutes. Supernatant was extracted with 6 ml methylene chloride for 20 minutes. The organic phase was then evaporated under nitrogen gas. The residues after evaporation were reconstituted with 100 μl of acetonitrile/water (50:50) and centrifuged at 16,000×g at 4° C. for 10 minutes. Finally, 50 μl of supernatant from each sample was injected onto a Waters XTerra MS C18 Column (2.1×150 mm, 3.5-μm particle size; Waters, Milford, Mass.) and elute with mobile phase containing acetonitrile/water [50:50 (v/v)] at flow rate of 0.2 ml/min. The UV absorbance of the eluents was monitored at 270 nm. Calibration standards were prepared in control mouse plasma with the compound concentrations ranging from 0.5 to 50 μg/ml. The recoveries of the Compound 1 over the calibration range were from 58.4 to 98.8%. The intra- and inter-day coefficients of variation of the assay were 11.6 and 7.8%, respectively, at 0.5 μg/ml (limit of quantitation, LOQ), and 12.6 and 11.8%, respectively, at 50 μg/ml. The plasma concentration-time data was analyzed by modeling using WinNonlin. One compartment model was chosen for all dose levels tested.

Based on the results from this study, the following conclusions may be obtained. First, volume distribution of Compound 1 in mouse, which is close to 3,000 ml/kg, is relatively large as compared with total body water of 725 ml/kg and total plasma volume of 50 ml/kg in mouse. Secondly, Compound 1 is extensively cleared in mouse. Since the clearance (118.8 ml/min/kg) is greater than mouse liver blood flow (90 ml/min/kg), the organ other than liver such as kidney also plays important role in Compound 1 elimination. Thirdly, Compound 1 is orally bio-available with oral bioavailability of 86% in mouse by assuming linear pharmacokinetics at dose levels tested. The unexpected high oral bioavailability of Compound 1 also suggests that liver is not the major elimination organ for Compound 1 in mouse. The concentration—time profile of Compound 1 after subcutaneous and oral dosing is shown in FIG. 14.

Experiment 14: Compound 4 was Not Found to Show Any Detectable Ocular Toxicity in Rats.

To test the potential ocular toxicities of Compound 4, normal rats at age of 8 weeks received an intravitreal injection of a high dose of Compound 4, 2 μg/eye (5 μl/eye of 0.4 mg/ml in BN rat serum) or an equal amount of BN rat serum as the vehicle control. Prior to study initiation, and after weeks 1, 2, 3, and 4 following the injection, visual function was evaluated by ERG recording. ERG recording showed no detectable change in the a-wave and b-wave amplitudes in Compound 4-injected rats compared to vehicle-injected eyes (FIG. 16 and FIG. 19A-C).

Possible toxicities of CLT-033 were also examined using pathohistological examination 4 weeks after the drug administration. Retinal cross sections stained with H&E were examined under a light microscope. No apparent morphological change or immunoresponse was found in the retinas treated with 2 μg/eye Compound 4 (5 μl/eye of 0.4 mg/ml), compared with the contralateral retina treated with the vehicle (FIG. 19D).

Discussion

Structure-activity-relationship studies showed that substituting the glutaramide ring of thalidomide with an aromatic group leads to active analogs. Specifically, replacing the glutaramide ring with 2, 6-diisopropylaniline yielded more active anti-angiogenic analogs—Compounds 1, 2, and 4.

In vitro screening using endothelial cell proliferation assay has demonstrated that three of the compounds, Compounds 1, 2, and 4 have potent anti-proliferative activities, as they selectively inhibited HUVEC and BRCEC growth with an IC₅₀ of <3.3 μM, which was substantially lower than that of thalidomide, and existing thalidomide analogs Actimid and Revimid (IC₅₀>100 μM). In addition, the thalidomide analogs did not inhibit the growth of non-endothelial cells, such as pericytes (IC₅₀>32 μM), suggesting that the inhibition to endothelial cell growth is cell type-specific rather than a result of non-specific cytotoxicities. One of the thalidomide analogs, Compound 4 displayed potent effects on growth of HUVECs and BRCECs (IC₅₀<3.3 μM), the migration of HUVECs (IC₅₀ of <1 μM), the tube formation of HUVECs, and vascular formation in the CAM assay (ED₅₀=6.5 μg/embryo). The anti-angiogenic effect of Compound 4 was also demonstrated in the OIR model, a commonly accepted model for retinal NV and for proliferative diabetic retinopathy.

The effects of thalidomide and novel analogs on retinal vascular leakage and NV have been compared in OIR and STZ-diabetic rats. The STZ-diabetic rats are a widely used model of experimental diabetes since the diabetic rats develop background diabetic retinopathy including vascular leakage. The OIR model is also shown to develop abnormal vascular leakage in the retina. The experimental results showed that the novel thalidomide analogs had significantly more potent effects on retinal vascular leakage than thalidomide in both animal models.

Compound 4 and thalidomide, at a single dose of 1.0 μg/eye, reduced retinal vascular leakage by 40% and 18% respectively when compared with vehicle control in OIR rats, in STZ-lnduced diabetic rats, Compound 4, thalidomide and Compound 1 at a single dose of 1.0 μg/eye reduced retinal vascular leakage by 100%, 77% and 61%, respectively, when compared with vehicle control. Twenty-four and 48 hours after a single administration, Compound 4 completely blocked retinal vascular leakage induced by diabetes. Compound 4 reduced retinal vascular leakage in a dose-dependent manner. These results indicate that Compound 4 has a potent effect on reduction retinal vascular leakage not only in the OIR model but also in the STZ-diabetes model, compared to thalidomide.

The regulatory effect of Compound 4 on VEGF expression in the retina of OIR rats has also been investigated with results showing that Compound 4 down-regulates the expression of VEGF. This suggests that Compound 4 targets signaling from VEGF.

The experiments have shown that Compound 4 inhibits cell proliferation in HUVEC and BRCEC, but not in non-endothelial cells, suggesting that its effect is endothelial cell-specific. Compound 4 was chosen to assess the potential ocular toxicity in rats. ERG recording and histopothological examination both demonstrated that Compound 4, at a single high dose, does not result in detectable changes in the ocular function and morphology in rats. The results imply that Compound 4 lacks significant toxicities at doses required for its anti-angiogenic activities.

These experiments have shown that a low dose of Compound 4 can inhibit NV. The more potent antiangiogenic effects of Compound 4 suggest that low doses of the compound are required to achieve inhibition of NV, and are therefore less likely to cause side effects.

Proteinuria in diabetic nephropathy is another type of vascular leakage. Compound 4 may also be applied to treat proteinuria due to its effect in reducing leakage of macromolecues out of blood vessels. Vascular leakage is an essential step in tumor metastasis. Blockage of vascular leakage of tumor vessels is also expected to have beneficial effect in solid tumor treatment.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

is should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicants).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonable expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A compound represented by a general structure: 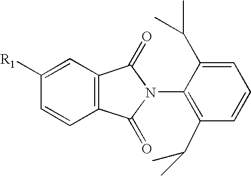
wherein R₁ is selected from a group comprising of hydroxy, hydrogen, and amino.

2. A composition for treating vascular abnormalities in a patient, said composition comprising a compound of a general structure: 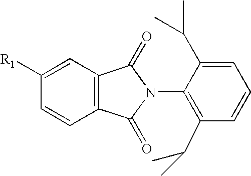
wherein R₁ is selected from a group comprising of hydroxy, hydrogen, and amino.

3. The composition of claim 2 wherein the vascular abnormality comprises at least one of neovascularization and vascular leakage.

4. The composition of claim 2 further including at least one agent.

5. The composition of claim 4 wherein the agent is a carrier, solubilizing agent, inert filler, diluent, excipient, or flavoring agent.

6. The composition of claim 2 wherein R₁ is an amino.

7. A method for treating vascular abnormalities in a patient, said method comprising administering to the patient a therapeutically effective amount of a composition, wherein the composition includes a compound of a general structure: 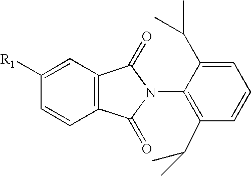
wherein R₁ is selected from a group comprising of hydroxy, hydrogen, and amino.

8. The method of claim 7 wherein the vascular abnormality comprises at least one of neovascularization and vascular leakage.

9. The method of claim 7 wherein the treatment includes suppressing HIF-1α.

10. The method of claim 7 wherein the treatment includes suppressing VEGF.

11. The method of claim 7 wherein the composition further includes at least one agent.

12. The method of claim 11 wherein the agent is a carrier, solubilizing agent, inert filler, diluent, excipient, or flavoring agent.

13. The compound of claim 7 wherein R₁ is an amino.

14. A method for synthesizing a compound of a general structure: 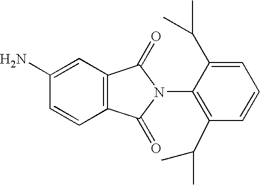
said method comprising providing (2,6-diisopropylphenyl)-5-amino-1H-isoindole-1,3-dione and refluxing the (2,6-diisopropylphenyl)-5-amino-1H-isoindole-1,3-dione with H₂, Pd/C, and acetone.

15. A method for synthesizing a compound of a general structure: 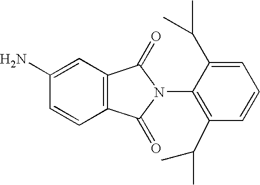
said method comprising providing 5-nitrophthalic anhydrides and 2,4-diisopropylaniline, refluxing 5-nitrophthalic anhydrides and 2,4-diisopropylaniline with acetic acid to form a (2,6-diisopropylphenyl)-5-amino-1H-isoindole-1,3-dione product, and refluxing the (2,6-diisopropylphenyl)-5-amino-1H-isoindole-1,3-dione product with H₂, Pd/C, and acetone.
